(19) United States
(12) Patent Application Publication
(10) Pub. No.: US 2025/0265828 A1
(43) Pub. Date: Aug. 21, 2025

(54) METHOD FOR VALIDATING PROCESSING DATA, METHOD FOR PROVIDING A MODEL THAT IS TRAINED BY MACHINE LEARNING, PROCESSING ENTITY, COMPUTER PROGRAM, AND DATA MEDIUM

(71) Applicant: Siemens Healthineers AG, Forchheim (DE)

(72) Inventors: Philipp Roser, Erlangen (DE); Markus Kowarschik, Erlangen (DE); Michael Manhart, Fürth (DE)

(21) Appl. No.: 19/054,848

(22) Filed: Feb. 15, 2025

(30) Foreign Application Priority Data
Feb. 15, 2024 (DE) 10 2024 201 389.7

(51) Int. Cl. G06V 10/776; G06V 10/75; G06V 10/82

(52) U.S. Cl. CPC G06V 10/776; G06V 10/751; G06V 10/82; G06V 2201/03

(57) ABSTRACT

A method for validating processing data includes receiving original data that is based on a medical image data acquisition. The processing data, which is based on application of a main processing algorithm to the original data, is received or determined. Comparison data that is or is based on the processing data is compared with reference data that is or is based on application of a reference processing algorithm to the original data using a comparison algorithm to determine a comparison result. A trigger condition, fulfillment of which depends on the comparison result, is evaluated, and when the trigger condition is fulfilled, a notification is output, a predetermined acquisition parameter is modified for a subsequent image data acquisition, and/or new processing data is provided. Either the reference data is used as new processing data, or the new processing data is determined by applying an alternative processing algorithm to the original data.

This application claims the benefit of German Patent Application No. DE 10 2024 201 389.7, filed on Feb. 15, 2024, which is hereby incorporated by reference in its entirety.

### BACKGROUND

The present embodiments relate to a method for validating processing data, a method for providing a model that is trained by machine learning, a processing entity, a computer program, and a data medium.

In the context of medical image data acquisition, before any evaluation that may be carried out, for example, by a medical specialist or even automatically or semiautomatically, raw image data that has been acquired may be processed by a processing algorithm, for example, in order to reconstruct three-dimensional or four-dimensional image data sets from individual projection recordings and/or in order to provide noise suppression, contrast enhancement, background suppression, accentuation of edges and/or vascular structures, suppression of artifacts (e.g., metal artifacts), etc.

As a result of such processing, the ease with which relevant structures and features may be identified may be greatly increased in comparison with the original data. At the same time, such processing may, however, lead to a distortion of the resulting processing data in some circumstances. For example, strong filtering or the like may result in the removal of structures that are actually depicted in the raw image data (e.g., fine vessels), as well as noise. For example, as a result of highly complex reconstruction algorithms or an algorithm for accentuating edges or vascular structures, artificial features that are not present in the raw image data may also be generated in the processing data. These may resemble anatomical features or lesions, medical devices, or the like, and consequently, in some circumstances, result in an erroneous assessment by a medical specialist or an evaluation algorithm.

Users of medical image data are therefore to be aware of this problem and check the plausibility of the processing result based on their experience. Alternatively, it is possible to restrict processing algorithms or to adapt the parameterization of algorithms in order to minimize the risk of distortion, whereby, however, the achievable image quality is typically reduced since, for example, only very weak noise suppression may be carried out.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, processing of medical image data is further improved, such as with the intention of avoiding or at least reducing the above cited disadvantages of the existing processing.

In one embodiment, a computer-implemented method for validating processing data includes receiving original data that is based on a medical image data acquisition, receiving or determining the processing data that is based on an application of a main processing algorithm to the original data, and comparing comparison data that is the processing data or is based on the processing data with reference data that is the original data or is based on an application of a reference processing algorithm to the original data using a comparison algorithm in order to determine a comparison result. The computer-implemented method also includes evaluating a trigger condition with fulfillment that depends on the comparison result, and if the trigger condition is fulfilled, outputting a notification to a user, and/or modifying a predetermined acquisition parameter for a subsequent image data acquisition, and/or providing new processing data, either using the reference data as new processing data or determining the new processing data by applying an alternative processing algorithm to the original data.

Using the comparison algorithm or the trigger condition that depends on the comparison result thereof, deviations that may occur in the context of the image processing may be identified automatically with good accuracy. For example, it is possible to identify a suppression of features that are actually present and/or a generation of artificial features by the main processing algorithm.

The fulfillment of the trigger condition may indicate an unacceptable deviation between the comparison data and the reference data. In this case, the processing data may be considered as validated processing data if the trigger condition is not fulfilled. If the trigger condition is fulfilled, the user (e.g., medical staff) may be notified, for example, that the processing data or comparison data appears to be distorted. In this case, the user may check the raw data by, for example, evaluation to determine whether a distortion is actually present and/or select another main processing algorithm or parameterize differently a basic algorithm that implements the main processing algorithm in order to prevent such a distortion.

In order to further simplify the task for the user, it may be appropriate, if an unacceptable deviation is identified or the trigger condition is fulfilled, additionally or alternatively to directly provide or further process new processing data. In this context, the reference processing algorithm or the alternative processing algorithm may, for example, perform processing that modifies the original data less extensively than the main processing algorithm (e.g., using weaker filtering and/or background suppression).

An adaptation of the parameterization of a medical image data acquisition may serve, for example, to minimize the exposure of the patient due to the measurement. In the case of x-ray imaging, for example, the As Low As Reasonably Achievable (ALARA) principle may be followed (e.g., the lowest possible x-ray dosage that is nonetheless appropriate for an imaging task may be applied to a patient). A dynamic parameterization of the image data acquisition may, however, also be appropriate for other imaging modalities (e.g., in the case of magnetic resonance imaging, in order to shorten a required imaging time as far as possible).

A change of the acquisition parameter in a first direction may, for example, result in a better image quality (e.g., a better signal-to-noise ratio), but at the same time, in an increase of the x-ray dosage and/or the measurement time or the patient exposure generally. Conversely, a change of the acquisition parameter in the opposite direction may reduce the patient exposure but nonetheless result in a lower image quality. The aim of dynamic parameter adaptation may be to keep the image quality at a required level while at the same time minimizing the exposure of the patient.

An identified unacceptably large deviation between the comparison data and the reference data in this context may indicate that the quality of the original data is inadequate. If the main processing algorithm performs, for example, merely a very weak noise suppression (e.g., smoothing by a narrow filter kernel), and such a minor modification already causes, for example, anatomical features, medical devices, or the like apparently depicted in the original data to be absent from the processing data, this may indicate, for example, that the signal-to-noise ratio concerned does not allow robust evaluation of the original data or that robust evaluation is not possible due to other interference (e.g., stripe artifacts that may occur in an image reconstruction when x-ray dosages are too low). In such cases, it may be appropriate to modify a predetermined acquisition parameter for the subsequent medical imaging (e.g., an x-ray dosage that is applied, an x-ray tube voltage, and/or a measurement time). Such an embodiment of the method may therefore also be considered as a method for the open-loop or closed-loop control of medical imaging (e.g., for adjusting the dosage that is used during x-ray imaging). The subsequent image data acquisition may be, for example, a subsequent image data acquisition based on fluoroscopy or generally further imaging of the same patient using the same medical imaging apparatus.

A modification of the predetermined acquisition parameter may, however, also be appropriate, or the or a further trigger condition may alternatively or additionally be fulfilled, if a measure of the deviation between the comparison data and the reference data, this being indicated for example by the comparison result, does not reach a limit value or if the comparison data and the reference data are all too similar. If, for example, a noise reduction that is performed as a main processing algorithm only results in an insignificant change of the original data, this may indicate that sufficiently good imaging would already be possible using an acquisition parameter that differs from the predetermined acquisition parameter and that would result in less exposure of the patient and/or a shorter measurement time. For example, the x-ray dosage or a tube voltage may be reduced in this case, or a tube parameter of an x-ray tube generally may be modified. With regard to magnetic resonance imaging, it is possible in this case to adapt, for example, sequence parameters in order to achieve less dense sampling of the k-space and thereby reduce the measurement time.

In comparison with known adjustment approaches that are, for example, based on an approximately determined noise component in the image or in a specified image section, the options explained for modifying a predetermined acquisition parameter may, using a suitable comparison algorithm that is able, as explained in greater detail below, for example, to identify when relevant features (e.g., anatomical features and/or depictions of medical devices) are distorted, use implicit prior knowledge relating to the object that is to be depicted, without requiring an exact model of the depicted region of the patient to be present. The resilience and accuracy of dynamic parameterization is further improved thereby.

Instead of an automated adaptation of the predetermined acquisition parameter or in addition to this, it is possible to notify the user accordingly, whereupon the user may alter the corresponding parameter manually, for example.

If it is intended to fulfill a plurality of the purposes explained above using the exemplified method, it may be appropriate to use separate instances of the method for the various purposes. It is possible to perform these instances in parallel or sequentially based on the same original data, for example. The instances of the method may differ from each other with respect to, for example, the main processing algorithms and/or trigger conditions that are used.

For example, a relatively mild de-noising may be used as a main processing algorithm to identify an x-ray dosage that is too low, while a stronger de-noising may be used as a main processing algorithm to provide processing data for further processing. It is then possible to check whether the stronger de-noising results in a distortion of the original data. The comparison result thus determined may optionally then be used additionally in a further trigger condition, using which the presence of all too small differences is identified in order to identify an unnecessarily high image quality and in this case to reduce the x-ray dosage or the like for the subsequent image data acquisition.

Use may be made of the same comparison algorithm in the various instances of the method. It is nonetheless possible in principle to use differing comparison algorithms.

An image data acquisition may, for example, be an acquisition of raw data from which an image data set is initially reconstructed by processing (e.g., the acquisition of magnetic resonance signals).

The original data and optionally the processing data may be received from, for example, an external apparatus, an internal data store, or a software module. The determination of the original data or the processing data may take place outside of the method of the present embodiments or alternatively also as a method act of this method.

The main processing algorithm may be used to improve an image and/or change an image impression. It is additionally or alternatively possible for the original data to include a plurality of two-dimensional image data sets. The main processing algorithm is or includes a reconstruction of a three-dimensional or four-dimensional image data set from these two-dimensional image data sets.

A main processing algorithm that is used for image improvement may be used, for example, for noise suppression, contrast enhancement, background suppression, and/or accentuation of edges and/or vascular structures and/or for artifact reduction (e.g., for the suppression of metal artifacts). Additionally or alternatively, the main processing algorithm may implement the production of a specific image impression desired by the user. For example, different users may prefer different degrees of background suppression and/or noise reduction when evaluating medical image data.

In the context of reconstruction, the main processing algorithm may reconstruct a computed tomography from a plurality of projection images. The plurality of projection images may be based on x-ray imaging, for example. For example, reconstruction of a conical-beam computed tomography is possible. The reconstruction may take place as a filtered back projection, a flow-restricted time-resolved filtered back projection, or as an iterative reconstruction.

A four-dimensional data set may be understood to be, for example, a time-resolved three-dimensional data set, as determined, for example, in the context of time-resolved three-dimensional angiography.

For example, the reference processing algorithm and/or the alternative processing algorithm may also be used for image improvement and/or for the reconstruction of a three-dimensional or four-dimensional image data set.

The main processing algorithm and the reference processing algorithm and/or the alternative processing algorithm may be implemented by respectively differing parameterization of a basic processing algorithm.

For example, these algorithms may differ from each other with respect to a filter parameter that is used, for example, with respect to the critical frequency of a low-pass filter for noise suppression, or with respect to a filter kernel that is used. It is thus possible for the different algorithms to result in different strengths of filtering (e.g., a stronger filtering typically providing better noise suppression but potentially also causing features that are actually present in the original data to be omitted or at least heavily suppressed in the processing data). It may therefore be advantageous to reduce the strength of the filtering in the reference processing algorithm and/or the alternative processing algorithm relative to the main processing algorithm (e.g., use a higher critical frequency of a low-pass filter or a narrower filter kernel).

The choice of the filter parameter may also decide, additionally or alternatively, whether and to what extent anisotropic filtering is to take place. Using anisotropic filtering, it is actually possible to achieve a sharper representation of edges and/or vascular structures with similarly good noise suppression. However, since anisotropic filtering may potentially cause a distortion of features, it may be advantageous to use isotropic filtering or less anisotropy in the reference processing algorithm or in the alternative processing algorithm than in the main processing algorithm.

Alternatively and additionally, it is possible using a different parameterization of the basic processing algorithm, for example, to set a degree of edge sharpness and/or background suppression. A strong edge sharpness and/or background suppression may facilitate the analysis of the processing data by a user or a subsequent evaluation algorithm or result in a preferable image impression. However, since such processes may potentially result in a suppression of relevant features and/or in the development of artifacts that a user or evaluation algorithm may potentially identify as a medical device, anatomical feature, or the like, it may be advantageous to reduce the degree of edge sharpness or background suppression in the reference processing algorithm or in the alternative processing algorithm in comparison with the main processing algorithm.

If the main processing algorithm is or includes a reconstruction of a three-dimensional or four-dimensional image data set from two-dimensional image data sets, for example, a filter kernel that is used in the context of a filtered back projection may be selected or adapted when parameterizing the basic processing algorithm, or for example, parameters of an iterative reconstruction may be adapted.

This provides, for example, that reconstructions may be performed using different parameterizations in the context of the main processing algorithm and the reference processing algorithm, whereupon the differing reconstruction results may be directly compared in the three-dimensional or four-dimensional space.

It is, however, also possible, for example, to initially perform a forward projection based on these differing reconstructions, so that a comparison of forwards projections by the comparison algorithm may then be used.

The cited parameters and other parameters with respect to which the cited algorithms may differ from each other may be, for example, parameters that may be set by a user in the context of image processing, or may be modified by a user by selecting various parameter sets, in order to produce a desired image impression.

It is, however, also possible for the reference processing algorithm and/or the alternative processing algorithm to be based on a different processing approach than the main processing algorithm. For example, at least one of the cited algorithms may be implemented using a model that has been trained by machine learning, and at least one other of the cited algorithms may be manually implemented or parameterized. Models that have been trained by machine learning may actually often achieve a particularly good image quality or a particularly advantageous image impression. It is, however, possible in individual cases for significant distortions of the resulting processing data to occur when such a trained model is applied. Using the method of the present embodiments, these cases may nonetheless be robustly identified by a comparison with the original data and/or by using a reference processing algorithm. This is implemented manually, for example. In the event of such a distortion, a notification or fall-back on the new processing data may therefore take place. In the case of a reconstruction likewise, it is also possible, for example, for one of the algorithms to be based on an iterative reconstruction and at least one other of the algorithms to be based on a filtered back projection.

As explained above, the original data may include a plurality of two-dimensional image data sets, the processing data taking the form of a three-dimensional or four-dimensional image data set that is reconstructed from these two-dimensional image data sets by the main processing algorithm. In this case, the comparison data may be or include two-dimensional image data sets that are determined by a respective forward projection of this three-dimensional or four-dimensional image data set.

In cases where the main processing algorithm is used for the reconstruction of a three-dimensional or four-dimensional image data set, as an alternative or in addition to the previously explained comparison of various parameterized reconstructions, it may be appropriate in the context of the comparison algorithm to check a consistency of the reconstructed image data set with the original two-dimensional image data sets or generally with two-dimensional reference data that may be produced by filtering or other preprocessing of the two-dimensional image data sets. For example, in the context of a three-dimensional angiography that is acquired by computed tomography (e.g., by conical-beam tomography), it is possible to check whether or to what extent the distribution of the contrast agent in the vascular system in the reconstructed four-dimensional image data set is consistent with the depiction of this distribution in the individual projection images that have been acquired. In order to achieve this, for example, the three-dimensional image data set corresponding to the time point of the recording of the respective projection image may be interpolated or selected from the four-dimensional image data set and forward projected according to the recording geometry of the respective projection image in order to provide the comparison data.

The comparison algorithm may be implemented by applying an evaluation algorithm to the comparison data in order to identify segments of the comparison data that depict a relevant anatomical feature and/or a medical device in each case, and to the reference data in order to identify segments of the reference data that depict a relevant anatomical feature and/or medical device in each case. The comparison result may then depend on a comparison of the number and/or the positions and/or the dimensions of the segments in the comparison data with the number and/or the positions and/or the dimensions of the segments in the reference data. Alternatively or additionally, it is possible to determine a boundary outline for the respective segment both in the reference data and in the comparison data, the comparison result depending on a comparison of the positions and/or the dimensions of the boundary outlines in the comparison data with the positions and/or the dimensions of the boundary outlines in the reference data.

In other words, it is possible to check whether the comparison data shows the same relevant regions as the reference data. In the simplest case, only the number of segments identified in the reference data and in the comparison data are compared as part of this activity. However, in order that a truncation of a vessel that actually extends further, or similar changes that may potentially be caused by the application of the main processing algorithm, may be identified more effectively, it may be appropriate additionally or alternatively to also consider the sizes or the positions of the various segments. The use of, for example, rectangular or cuboid boundary outlines as opposed to a comparison of irregularly shaped segments may make the comparison of the positions or the dimensions easier and/or more reliable.

The comparison takes place, for example, between data that are registered to each other, so that the positions and dimensions of the segments or of boundary outlines containing the respective segment (e.g., boundary rectangles or boundary cuboids) may be compared directly.

The anatomical features and/or medical devices that are to be taken into consideration are, for example, definitively predetermined. This provides that it is possible, for example, to definitively predetermine which segments are be taken into consideration during the evaluation of the trigger condition. A number of algorithms for segmenting medical image data or specific anatomical features and/or medical devices in medical image data are known from the field of medical image data processing, and these may be used as an evaluation algorithm or as part of the evaluation algorithm in the method of the present embodiments.

In an embodiment, the comparison algorithm is or includes a model that is trained by machine learning. The model that is trained by machine learning may implement the previously explained evaluation algorithm, for example. Using machine learning, it is also possible reliably to solve complex segmentation and/or classification tasks in medical image data.

For example, the model that is trained by machine learning may be based on supervised learning with backpropagation, with training data sets that include a desired result in addition to the data that is to be segmented. For example, the desired result may be or include a segmentation or classification of the data that is to be segmented, which has been performed manually by at least one expert.

As explained in greater detail below, a model that is trained by machine learning may, however, also be trained to immediately identify whether a relevant change is present, or a plurality of models may be trained to check the processing data with respect to a specific change type in each case.

In general, a model that is trained by machine learning emulates cognitive functions that humans associate with the mind of other humans. By training based on training data, for example, the trained model is able to adapt to new circumstances and to identify and extrapolate patterns. A “model that is trained by machine learning” may also be referred to as a “trained function”.

In general, parameters of the model may be adapted by training. For example, supervised learning, semi-supervised learning, unsupervised learning, reinforcement learning, and/or active learning may be used. Representation learning (e.g., “feature learning”) may also be used. For example, the parameters of the trained model may be iteratively adapted by a plurality of training steps. For example, a predetermined cost function may be minimized in the training. When training a neural network, for example, backpropagation may be used for the training.

A model that is trained by machine learning may be based on, for example, a neural network, a support vector machine (SVM), a decision tree, and/or a Bayes network, and/or on k-means clustering, Q-Learning, genetic algorithms, and/or rules of assignment. For example, a neural network may be a deep neural network, a convolutional neural network (CNN), or a deep CNN. Further, the neural network may be an adversarial network, a deep adversarial network, and/or a generative adversarial network (GAN).

The training of the model that is trained by machine learning may takes place outside of the computer-implemented method. For example, the computer-implemented method may be performed in the context of the application of medical imaging and/or an evaluation of image data from such imaging (e.g., in a clinic or by an independent doctor). The training of the model may be independent of this with respect to place and time, and may be performed by other people (e.g., by a manufacturer of an imaging apparatus concerned during the course of its manufacture or development).

The comparison algorithm may include at least a first model and a second model that are trained by machine learning and that, in each case, process the comparison data and the reference data as input data. The first model that is trained by machine learning may determine a first intermediate result that relates to the presence and/or the extent of a deviation of a first deviation type between the comparison data and the reference data. The second model that is trained by machine learning may determine a second intermediate result that relates to the presence and/or the extent of a deviation of a second deviation type, this differing from the first deviation type, between the comparison data and the reference data. The comparison result may then be dependent on the first intermediate result and the second intermediate result.

It is also possible for more than two deviation types to be identified by models that are trained to identify the respective deviation type. This provides that the comparison algorithm may additionally include at least one further model that is trained by machine learning and by which a respective further intermediate result is determined. This further intermediate result describes in each case whether a deviation of the respective further deviation type between the comparison data and the reference data is identified, and the fulfillment of the trigger condition depends additionally on the respective further intermediate result.

The trigger condition may already be fulfilled if, for example, one of the trained models in use identifies a deviation between the first input data and the second input data.

An identification of different deviation types by separate trained models may allow the use of a simpler algorithm that forms the basis of the respective model. If the model is based on a neural network, for example, this may have fewer nodes or layers or associations than would be required for a model that is able by itself to identify all relevant deviation types. The training of separate models to identify different deviation types may also allow faster training and fewer required training data sets. Alternatively, it is nonetheless entirely possible to use a single model that is trained by machine learning in order to identify all relevant deviation types.

As explained above, the training of the models that are trained by machine learning may take place outside of the claimed computer-implemented method. The first model and/or second model that is trained by machine learning may be trained by supervised learning, for example. For this purpose, it is possible to use training data sets that each include pairs of source or reference data and comparison data. In addition to this, for at least one deviation type, the presence and/or the extent of the deviation of the respective deviation type may be stored in the training data set as target information. The pairs may be derived from earlier medical imaging operations and/or from a simulation, and the target information may be predetermined, for example, based on a manual assessment of the respective pair by medical specialists. Supervised learning may take place in a customary manner based on these training data sets (e.g., by minimizing a cost function using backpropagation, such as by a gradient descent method).

A discriminator may be used as the model that is trained by machine learning or as at least one of the models that is trained by machine learning. The parameterization of the discriminator is based on a training of a generative adversarial network that includes the discriminator. A generative adversarial network (GAN) includes a generator and a discriminator, and in this context, the generator creates synthetic data and the discriminator distinguishes between synthetic and real data. As a result of training the generator and/or the discriminator, the generator is configured to generate synthetic data that is erroneously classified as real by the discriminator. The discriminator is configured to distinguish between real data and synthetic data that is generated by the generator.

As explained above, the training of the model that is trained by machine learning may take place outside of the claimed computer-implemented method. The GAN generator that is used in the context of the training may be parameterized in the context of the training such that the GAN generator generates features with respect to which the discriminator (and hence the model that is trained by machine learning and used in the context of the comparison algorithm) is intended to detect differences that are as close as possible to reality. In terms of gaming theory, a GAN may be interpreted as a zero-sum game. The training of the generator and/or the discriminator may be based, for example, on the minimization of a cost function. The minimization of the cost function may be effected by a gradient descent method (e.g., using backpropagation). The generator and the discriminator may each be implemented as a convolutional neural network (CNN), for example.

In the context of training the GAN, the generator may be trained to generate, for example, a synthetic depiction that is parameterized by input data and is intended to depict, for example, at least one anatomical feature and/or at least one medical device with an identifiable depiction in the processing data that is to be checked by the comparison algorithm. As a result of the coordinated training of the discriminator and the generator, the discriminator is consequently trained to reliably identify both an absence and an erroneous depiction of these features, and is therefore highly suitable for identifying any suppression or distortion of corresponding features by the main processing algorithm.

It is alternatively also possible to use the main processing algorithm directly as a generator in the context of such training. The main processing algorithm may be definitively predetermined, for example, or trained as part of the training of the GAN (e.g., in order to train a filter algorithm using machine learning).

The comparison result may describe in each case the presence and/or the extent of a deviation of at least one of the following deviation types: an absence, interruption, and/or truncated representation of at least one vessel; and/or the absence of the depiction of a medical device; and/or the absence of the depiction of an anatomical feature in the comparison data relative to the reference data and/or on in the reference data relative to the comparison data.

An absence of the representation of a vessel, a medical device, or an anatomical feature in the comparison data relative to the reference data indicates a suppression of the respective representation (e.g., caused by filtering that is too strong). Filtering that is too strong or other types of representational change (e.g., over-aggressive suppression of an image background) may also cause an apparent truncation or interruption of a depicted vessel as a result of applying the main processing algorithm.

The opposite case, whereby features in the reference data appear to be absent or vessels appear to be truncated or interrupted relative to the comparison data, indicates that a feature that was not actually present in the reference data or original data has been added as a result of applying the main processing algorithm.

The exemplified deviations may be identified, for example, by the segmentation explained above. Additionally or alternatively, corresponding features and therefore likewise the cited differences or changes may also be identified using a suitable model that is trained by machine learning. For example, in the case of training data sets that include pairs of reference data and comparison data in each case, manual annotation by medical specialists may be used to indicate whether deviations or which of the cited deviations are present. Supervised learning may then be carried out in a known manner based on a plurality of these training data sets.

Since annotation of training data sets may be relatively resource-intensive, it is also possible as explained above to use a GAN in the context of the training in order to train a discriminator that is suitable for identifying the cited deviations. As a result of the interaction of generator and discriminator in the GAN, training success may already be achieved with considerably less training data, or the training success may be further improved with a given amount of training data. Further, annotation of training data is not required in this case, and unsupervised learning may take place.

In an embodiment of the method, it is also possible to receive earlier original data that is based on a previous medical image data acquisition that took place prior to the medical image data acquisition, the or an acquisition parameter for the or a subsequent image data acquisition being specified as a function of a further comparison result that is determined by the comparison algorithm by comparing current input data that corresponds to or is based on the original data with earlier input data that corresponds to or is based on the earlier original data.

Using the procedure described, changes of the image quality in successive image data acquisitions (e.g., during a fluoroscopy or in the case of consecutively acquired projection recordings in the context of computed tomography) may be identified and taken into consideration when specifying the acquisition parameter (e.g., an x-ray dosage, an x-ray tube parameter, or a measurement time). For example, in the context of dosage adjustment, the x-ray dosage may be progressively reduced in consecutive image data acquisitions until it is confirmed based on the further comparison result that at least one relevant feature is no longer depicted, or the like. An adaptation of the acquisition parameter may also be appropriate in order to compensate at least largely for a change in the x-ray attenuation of the patient when the recording geometry changes between the various image data acquisitions.

As explained above, the acquisition parameter in this context may also depend on the comparison result or may also depend exclusively on the further comparison result, for example, if the evaluation of the trigger condition as explained above serves exclusively for the purpose of notification and/or to provide new processing data.

The comparison information may, at least in the event that the trigger condition is fulfilled, relate to at least one segment of the comparison data in which the comparison data differs from the reference data. The notification includes segment information that relates to this segment. As a notification, a user may be shown, for example, the comparison data or the processing data, and the reference data or the original data. The at least one segment with respect to which the deviation was identified is highlighted in order to indicate the respective type and degree of deviation or distortion for the user.

Alternatively or additionally, the notification may include a recommendation for the choice of a suitable main processing algorithm or a recommended value for at least one parameter of the or a basic algorithm that forms the basis of the main processing algorithm. This may allow the user to continue with their choice of main processing algorithm or parameters, but nonetheless to receive a notification of a choice that is likely to be particularly advantageous.

The present embodiments further relate to a computer-implemented method for providing a model that is trained by machine learning and is intended for use as a comparison algorithm or as a sub-algorithm of the comparison algorithm in the computer-implemented method of the present embodiments for validating processing data. The computer-implemented method for providing a model that is trained by machine learning includes the following acts: receiving input training data that includes a plurality of training data sets, these in turn including training data that is based in each case on a medical image data acquisition and/or a simulation of a medical image data acquisition; training a model based on the input training data for the purpose of determining the model that is trained by machine learning; and providing the model that is trained by machine learning.

Various possibilities for performing such training are already explained above and therefore need not be repeated. The training data may be used, for example, as original or reference data in the context of the training. If processing data or comparison data is required in the context of the training, this may be determined based on the respective training data or provided directly as part of the respective training data set.

It is possible in general to realize unsupervised learning, for example, using the detector of a GAN as a trained model, or supervised learning, for example, for learning about deviations that have previously been classified manually or using other methods. The latter may be used, for example, for the separate training of the first trained model and the second trained model explained above. The embodiment possibilities explained above may be transferred with the same advantages to the computer-implemented method for providing a model that is trained by machine learning.

The present embodiments further relate to a processing entity that is configured to carry out the computer-implemented method of the present embodiments for validating processing data and/or the computer-implemented method of the present embodiments for providing a model that is trained by machine learning. The processing entity may take the form of suitably programmed data processing apparatuses, for example, or the cited functionality may alternatively be at least partly hard-wired. The processing entity may be integrated in a medical imaging apparatus (e.g., an x-ray apparatus or a magnetic resonance tomography system) or be developed separately from these. It may be implemented as a workstation computer, a server, or a cloud-based solution, for example.

The present embodiments further relate to a computer program having instructions that are configured to carry out the computer-implemented method of the present embodiments for validating processing data and/or the computer-implemented method of the present embodiments for providing a model that is trained by machine learning when executed on a data processing apparatus.

The present embodiments further relate to a data medium that contains the computer program of the present embodiments.

### BRIEF DESCRIPTION OF THE DRAWINGS

Further advantages and details of the invention are derived from the following example embodiments and the associated drawings, in which:

FIG. 1 schematically shows a sequence diagram of an example embodiment of a computer-implemented method for validating processing data;

FIG. 2 schematically shows an example embodiment of a processing entity;

FIGS. 3-5 schematically show sub-sequence diagrams of further example embodiments of the computer-implemented method for validating processing data;

FIG. 6 schematically shows a sequence diagram of an example embodiment of the computer-implemented method for providing a model that is trained by machine learning; and

FIGS. 7 and 8 schematically show possible structures of the model that is trained by machine learning.

### DETAILED DESCRIPTION

As explained in the introduction, it is possible for individual features to be suppressed or even for artificial new features to be generated when processing original data 2 from medical imaging, for example, as a result of filtering, reconstruction, contrast enhancement, etc. In order to prevent this from causing an erroneous evaluation of the image data, for example, it is appropriate to validate the processing results.

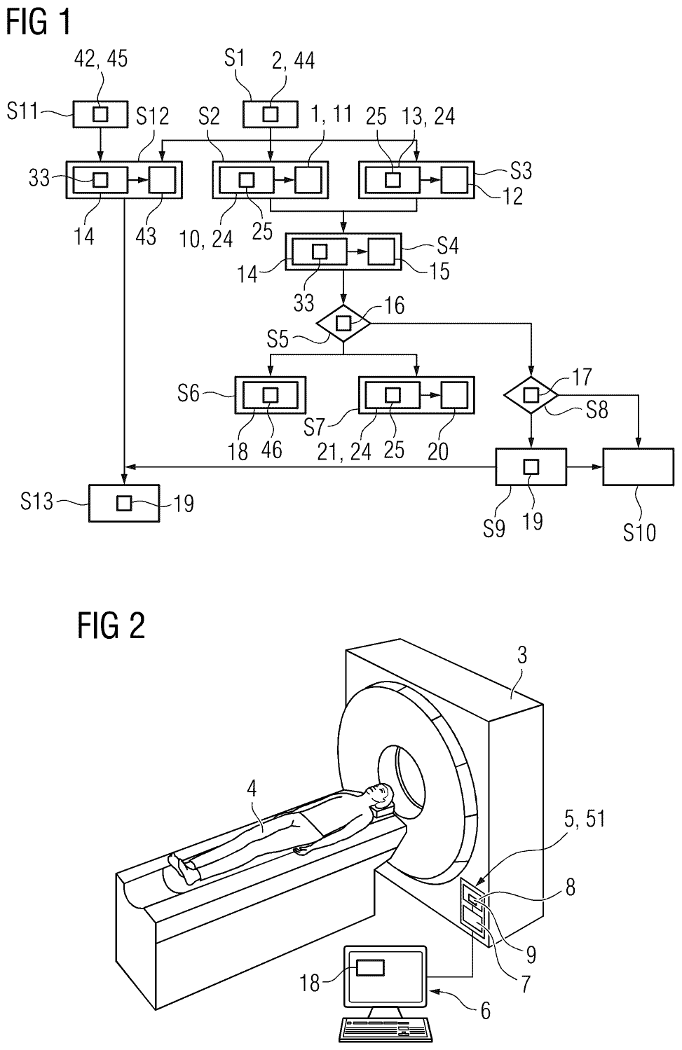

FIG. 1 shows a sequence diagram of a computer-implemented method for performing such a validation of processing data 1. Since the method illustrated in FIG. 1 is relatively complex, for better understanding, a brief overview of the central parts of the method is provided first, before the individual acts of the specific example are explained in detail.

In acts S1 and S2, the original data 2, which is based on a medical image data acquisition, and processing data 1 that is based on an application of a main processing algorithm 10 to the original data 1 are initially received.

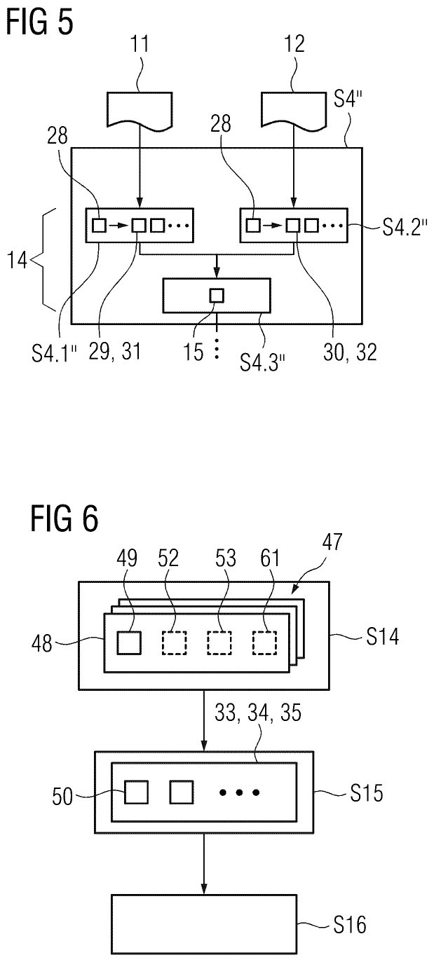

In act S4, a comparison then takes place between comparison data 11 that corresponds to the processing data 1 in the example and reference data 12 that is based on an application of a reference processing algorithm 13 to the original data 1 in the example. In modifications of the example embodiment shown, it would also be possible for the processing data 1 to be initially processed further before the comparison and/or for the original data 1 to be used directly as reference data 12. An example of this alternative embodiment is explained below with reference to FIG. 3.

The comparison result is evaluated in acts S5 or S8 by a respective trigger condition 16, 17. Depending on whether one or which of the trigger conditions 16, 17 is fulfilled, various actions are performed in acts S6, S7, S9, and S10. In this example, in act S6, a notification 18 is output to a user. In act S7, new processing data 20 is provided by applying an alternative processing algorithm 21 to the original data 2. In act S9, a predetermined acquisition parameter 19 is modified for a subsequent image data acquisition. In act S10, the validated processing data 1 is provided. In a modification of the determining procedure, it would also be possible in act S7 for the already known reference data 12 to be provided directly as new processing data 20.

In the following, the individual acts of the example embodiment shown in FIG. 1 are explained in greater detail. The following explanation assumes, for example, that the original data 2 is an individual two-dimensional image data set in each case (e.g., an x-ray recording). It is likewise assumed by way of example that the processing data 1 that is to be validated is based on a filtering of this original data 2. Alternatively or in addition to filtering, the main processing algorithm 10 may, however, also perform other processing acts for improving the image and/or changing an image impression. Examples of these were already discussed in the general part of the description.

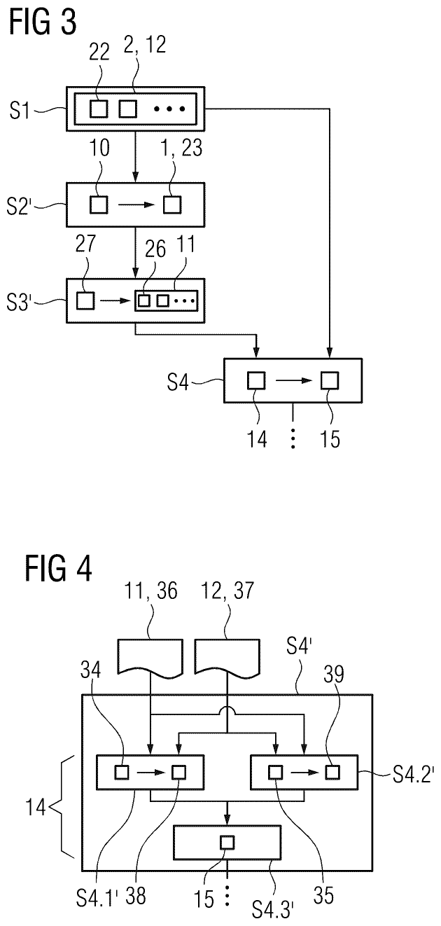

The explanation of the method also makes reference to FIG. 2, which illustrates an application scenario for the claimed method and an example implementation of a processing entity 5 that implements the method. In the example, the processing entity 5 is integrated in a medical imaging apparatus 3 (e.g., an x-ray apparatus, such as a computed tomography system). In the example, the processing entity 5 is implemented by a processor 7 of a freely programmable data processing apparatus 51 executing a computer program 9 that is stored in the memory 8 and having instructions implement the method acts. Various further implementation possibilities for suitable processing entities 5 were already discussed in the general part of the description.

In act S1, original data 2, which takes the form of two-dimensional x-ray images of the patient 4 in this example, is initially acquired by the medical imaging apparatus 3 and received by the processing entity 5. In alternative embodiments, it would also be possible already in advance, for example, to read original data that was acquired outside of the method (e.g., from a database).

In act S2, the original data 2 is processed by the main processing algorithm 10 in order to obtain the processing data 1. Alternatively, it would also be possible to perform the processing, for example, in a separate apparatus (e.g., in the workstation computer 6), and therefore, to receive the processing data 1 for validation by the processing entity 5 from the separate apparatus.

The main processing algorithm 10, in the same way as the reference processing algorithm 13 and the alternative processing algorithm 21 explained below, is implemented in the example by specifying at least one parameter 25 of a basic processing algorithm 24. The parameter has different values for the different cited algorithms. The parameter 25 may specify, for example, a critical frequency of a filter or the size and/or shape of a filter kernel for the filtering.

In act S3, the reference processing algorithm 13 is applied to the original data 2 in order to obtain reference data 12. As explained above, the reference processing algorithm 13 in the example corresponds to the main processing algorithm 10 apart from the choice of the parameter 25. The parameter 25 of the reference processing algorithm 13 is selected such that any distortion of relevant features in the reference data 12 may be ruled out. For example, a suitable choice of the parameter 25 may provide that only very weak filtering takes place, such that relevant features that may potentially be suppressed by the stronger filtering of the main processing algorithm 10 are reliably preserved. In alternative embodiments, it would also be possible to use the original data 2 directly as reference data 12.

In act S4, comparison data 11 that corresponds to the processing data 1 in the example but may in alternative embodiments also be derived therefrom by further processing is compared with the reference data 12 using the comparison algorithm 14 in order to determine a comparison result 15.

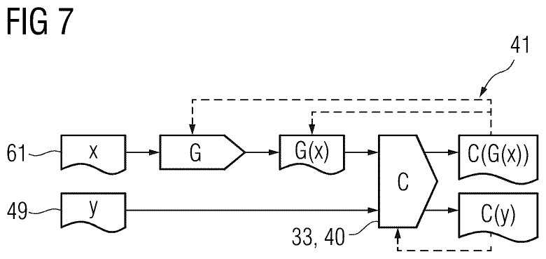

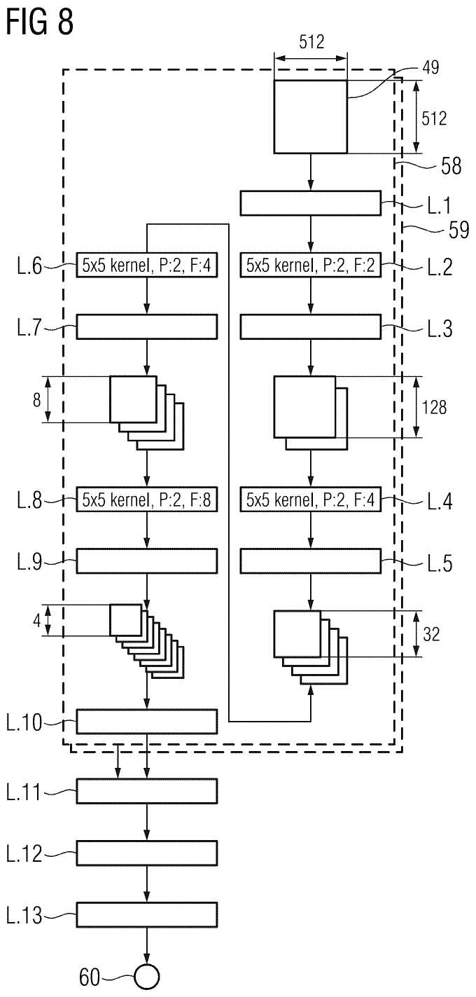

The comparison algorithm 14 is implemented in the example by a model 33 that is trained by machine learning. As explained below in more detail with reference to FIGS. 6 and 7, a discriminator 40 is used in the example as the model 33 that is trained by machine learning. The parameterization of the discriminator 40 is based on a training of a generative adversarial network 41 that includes the discriminator 40.

In the simplest case, the comparison information 15 may specify a degree of the deviation or a probability (e.g., normalized between 0 and 1) that the comparison data 11 differs from the reference data 12 in an unacceptable manner (e.g., to the extent that essential features are suppressed or distorted).

In act S5, a trigger condition 16 is then evaluated. The trigger condition 16 is fulfilled in the example if the comparison information 15 does not reach a limit value, and therefore, it may be assumed that the main processing algorithm 10 has distorted the original data 2 in an unacceptable manner.

In this case, a notification 18 is output to a user in act S6 (e.g., via the workstation computer 6). As explained in the general part, the notification 18 may include segment information 46 that may describe, for example, the segment of the processing data 1 in which the corresponding deviation occurs. In order to achieve this in the example shown, for example, the model 33 that is trained by machine learning may be configured and trained to additionally provide the segment information 46. Corresponding segment information 46 may be provided in a particularly simple and reliable manner if a segmentation takes place in the context of the comparison algorithm 14, as explained below with reference to FIG. 4.

In addition to this, in act S7, new processing data 20 is directly provided as a result of processing the original data 2 by the alternative processing algorithm 21. Using a suitable selection of the parameter 25, this may, for example, perform filtering at a strength that lies between the filter strength of the reference processing algorithm 13 and that of the main processing algorithm 10. It may thus be assumed, initially, with high probability, that the distortion of the processing data 1, which resulted in the fulfillment of the trigger condition 16 in step S5, is not present or is at least significantly less evident in the new processing data 20. It may optionally be possible, for example, to perform the previously explained validation for the new processing data 20 again. If this validation also fails, it would be possible, for example, to provide the original data 2 directly as new processing data 20 or to modify the parameter 25 again.

If the trigger condition 16 is not fulfilled, a check in act S8 ascertains whether the further trigger condition 17 is fulfilled. This further trigger condition 17 is configured to be fulfilled in the example if the comparison result 15 indicates an extremely small deviation between the comparison data 11 and the reference data 12, for example if the comparison result 15 does not reach a predetermined limit value. This indicates that, as a result of the main processing algorithm 10 or the de-noising that is implemented thereby in the example, only a negligibly small change of the original data is produced. This indicates that the image quality of the original data 2 is unnecessarily high for the given imaging task.

This provides that if the further trigger condition 17 is fulfilled, it is possible in act S9 to effect, for example, a reduction of an x-ray dosage and/or an off-load voltage, or generally a modification of at least one predetermined acquisition parameter 19 for a subsequent image data acquisition, since a somewhat lower signal-to-noise ratio may be permitted in this case. Therefore, an exposure of the patient 4 may be reduced.

Irrespective of whether the further trigger condition 17 is fulfilled, the now validated processing data 2 is provided in act S10 (e.g., whenever the trigger condition 16 is not fulfilled), so that the now validated processing data 2 may be displayed and/or further processed at the workstation computer 6, for example.

As explained above with reference to act S9, the comparison of various data by the comparison function 14 may also be appropriate for a dosage adjustment of an x-ray dosage or generally for the parameterization of a subsequent image data acquisition. Therefore, in the example shown in FIG. 1, acts S11 to S13 for adaptation of the acquisition parameter 19 are performed in parallel with the acts explained above.

In order to achieve this, previous original data 42 based on an earlier medical image data acquisition that took place before the medical image data acquisition is initially received in act 11. For example, the image data that was recorded in the context of a fluoroscopy may be read out from the memory 8.

In act S12, the comparison algorithm 14 is then used again to compare current input data 44 that corresponds to or is based on the original data 2 with previous input data 45 that corresponds to or is based on the previous original data 42. The resulting further comparison result 43 therefore indicates whether, for example, due to a dosage adaptation between the image data acquisitions and/or other influences, there is a danger that features are absent or distorted and/or that artifacts are present in the current original data due to an excessively low x-ray dosage. In act S13, the acquisition parameter 19 is then adapted as a function of this further comparison result 43 (e.g., in order to increase a dosage). This may take place additionally as a function of the value that was potentially predetermined by act S9 for the acquisition parameter 19.

For the sake of completeness, it should be noted that the procedure explained with reference to FIG. 1 may also be used in cases where the main processing algorithm 10 performs a reconstruction of three-dimensional or four-dimensional image data sets (e.g., in the context of a computed tomography (CT) or a time-resolved CT angiography). In this case, the reference processing algorithm 13 may likewise perform a corresponding reconstruction, though, for example, a different filter kernel than in the main processing algorithm 10 is used in the context of the reconstruction, or instead of an iterative reconstruction in the main processing algorithm 10, a reconstruction by back projection is used in the reference algorithm 13, etc. In the example shown in FIG. 1, the comparison algorithm 14 directly compares the three-dimensional or four-dimensional image data sets in this case.

A direct comparison of three-dimensional or four-dimensional image data sets by the comparison algorithm 14 potentially has considerable processing overheads. In addition, it is potentially necessary to process very large amounts of input data, so that if the comparison algorithm 14 is implemented as a neural network, for example, a very large number of input nodes and consequently in total very many layers and nodes per layer are required. This may result in a very large number of free parameters, whereby training of a corresponding algorithm is resource-intensive and typically requires very many training data sets.

In FIG. 3, the exemplary embodiment shown in FIG. 1 is therefore modified somewhat for the purpose of validating processing data 1 in the form of a three-dimensional or four-dimensional image data set 23, specifically by replacing acts S2 and S3 with acts S2′ and S3′ explained below. As schematically illustrated in relation to act S1, the original data 2 includes a plurality of two-dimensional image data sets 22, for example.

In act S2′, the reconstruction of the three-dimensional or four-dimensional image data set 23 from the two-dimensional image data sets 22 takes place first. When reconstructing a four-dimensional data set of a computed tomography-based angiography, for example, the reconstruction may only be approximate, and therefore, the validation of the processing data 1 is particularly relevant in this case.

In act S3′, a forward projection 27 then takes place in order to provide two-dimensional image data sets 26 as comparison data 11. If the processing data 1 is a three-dimensional image data set 23, such a forward projection 27 may take place directly. However, if the processing data 1 is a four-dimensional image data set 23, a three-dimensional image data set may first be determined (e.g., selected or interpolated from the four-dimensional image data set) for the respective time point at which the respective two-dimensional image data set 22 of the original data 2 was acquired, and this three-dimensional image data set may be forward projected.

In act S4, the comparison data 11 determined in act S3′ is then compared with reference data 12 that was directly formed by the original data 2 in the example shown. Alternatively, as explained previously in relation to FIG. 1, a reference processing algorithm may be used in order to perform, for example, a filtering of the image data sets 22. For example, the individual image data sets 26 of the comparison data may be compared with the individual image data sets 22 of the reference data 12 in this context.

The method may then be continued as explained above in relation to FIG. 1.

FIG. 4 shows an alternative embodiment of the comparison algorithm 14 in act S4′, which may replace act S4 from FIG. 1 or FIG. 3. Act S4′ includes a plurality of subacts.

In the first subact S4.1′, the comparison data 11 and the reference data 12 are processed as input data 36, 37 by a first model 34 that is trained by machine learning in order to determine a first intermediate result 38. This relates to the presence and/or the extent of a deviation of a first deviation type between the comparison data 11 and the reference data 12.

In the second subact S4.2′, the same input data 36, 37 is processed by a second model 35 that is trained by machine learning in order to determine a second intermediate result 38. This relates to the presence and/or the extent of a deviation of a second deviation type between the comparison data 11 and the reference data 12.

The first intermediate result 38 and the second intermediate result 39 specify a probability, normalized in each case between 0 and 1, that a deviation of a specific deviation type is present.

The intermediate results 38, 39 are then combined in the third subact 4.3′ to form the comparison result 15. In the simplest case, the comparison result 15 may be a list of the various intermediate results 38 and 39, in which case the trigger condition 16 may include, for example, a plurality of subconditions that may implement, for example, a limit value comparison per intermediate result 38, 39. The trigger condition 18 may then already be fulfilled if, for example, one of the subconditions is fulfilled. It may, however, also be appropriate to combine the various intermediate results 38, 39 to form a single value (e.g., by using the largest of the intermediate results 38, 39 as a comparison result 15 or by using the sum or a product of the intermediate results 38 and 39 as a comparison result 15). In this case, the trigger condition 18 may be implemented, for example, by a simple limit value comparison.

A suitable approach for training the first model 34 and the second model 35 that are trained by machine learning is explained below with reference to FIGS. 6 and 8.

FIG. 4 shows a further alternative embodiment of the comparison algorithm 14 in act S4″, which may replace act S4 in FIG. 1 or FIG. 3. Act S4″ includes a plurality of subacts.

In the subact S4.1″, an evaluation algorithm 28 is applied to the comparison data 11 in order to identify segments 29 of the comparison data 11 that depict a relevant anatomical feature and/or a medical device in each case. Since a number of suitable segmentation or evaluation algorithms are known, it is not intended to describe the segmentation in detail. Purely by way of example, a model that is trained by machine learning may be used for the purpose of segmenting or classifying the individual segments. It is, however, also possible to use known analytical algorithms (e.g., based on limit value comparisons, region growing, etc.).

In the subact S4.2″, the evaluation algorithm 28 is applied to the reference data 12 in order to identify segments 30 of the reference data 12 that depict a relevant anatomical feature and/or a medical device in each case.

In act S4.3″, the number and/or the positions and/or the dimensions of the segments 29 in the comparison data 11 may then be compared with the number and/or the positions and/or the dimensions of the segments 30 in the reference data 12 in order to determine the comparison result 15. For example, it is possible to check in this context whether all of the segments 29 that were identified in the comparison data 11 were also identified in the reference data 12 and vice versa.

As explained previously in the general part, in addition or as an alternative to the comparison of the segments 29, 30, it is also possible to compare boundary outlines 31, 32 that delimit these in order to determine the comparison result 15.

FIG. 6 shows a sequence diagram of a method for providing a respective model 33, 34, 35 that is trained by machine learning and may be used as a comparison algorithm 14 or sub-algorithm of the comparison algorithm 14 as explained above with reference to FIG. 1 or FIG. 4. By way of example, the training of the model 33 is initially explained here with additional reference to FIG. 7.

In act S14, input training data 47 that includes a plurality of training data sets 48, each including training data 49 that is based on a medical image data acquisition and/or a simulation of a medical image data acquisition, is received or provided.

In act S15, training of a model then takes place based on the input training data 47 in order to determine the model 33 that is trained by machine learning. In the example, a discriminator 40 having parameterization that is based on the training of a generative adversarial network 41 that includes the discriminator 40 is used as the model 33 that is trained by machine learning. In this case, the training data sets may additionally include training parameters 61 that may be converted by the generator G of the generative adversarial network 41 into synthetic output data G(x). This may describe, for example, the recording geometry for the respective training data 49 and patient parameters (e.g., the sex, the height, and the weight of the patient).

FIG. 7 shows an example data flow diagram of such a generative adversarial network 41. The generative adversarial network 41 is used to generate synthetic output data G(x) based on input data x. The output data G(x) is designed to be indistinguishable from real output data. In the example, the training parameters 61 are used as the input data x in the training. The training data (e.g., x-ray projection images) is used as the real output data y. The synthetic output data G(x) has the same structure as the real output data y, but the content thereof is not derived from real data.

The generative adversarial network 41 consists of a generator function G and a classification function C that are trained together. The classification function forms the discriminator 40 and, after it has been trained, may be used as the model 33 that is trained by machine learning in the method according to FIG. 1.

The task of the generator function G consists in delivering realistic synthetic output data G(x) based on input data x, and the task of the classification function C consists in distinguishing between real output data y and synthetic output data G(x). For example, the output of the classification function C is a real number between 0 and 1, which corresponds to the probability that the input value is genuine data; therefore, an ideal classification function would calculate an output value of C(y)≈1 for genuine data y and C(G(x))≈0 for synthetic data G(x).

In the context of the training process, the parameters of the generator function G are adapted such that the synthetic output data has the same properties as the real output data, so that the classification function C may no longer distinguish between real and synthetic data. At the same time, the parameters of the classification function C are adapted such that the classification function C distinguishes as efficiently as possible between real and synthetic data. The training in this case is based on pairs of input data x and the corresponding real output data y. In a single training step, the generator function G is applied to the input data x in order to generate synthetic output data G(x). Further to this, the classification function C is applied to the real output data y in order to generate a first classification result C(y). The classification function C is additionally applied to the synthetic output data G(x) in order to obtain a second classification result C(G(x)).

The adaptation of the parameters 50 of the generator G and the classification function C is based on the minimization of a cost function using the back propagation algorithm. In this embodiment variant, the cost function K_{C} for the classification function C is

K_{C}∝−BCE(C(y),1)−BCE(C(G(x),0),

where BCE designates the binary cross entropy, this being defined as

BCE(z,z′)=z′−log(z)+(1−z′)−log(1−z).

As a result of using this cost function, the cost function K_{C} that is to be minimized increases both in the event of the erroneous classification of real output data as synthetic (characterized by C(y)≈0) and in the event of the erroneous classification of synthetic output data as real (characterized by C(G(x))≈1).

The cost function K_{G} for the generator function G in the example is

K_{G}∝−BCE(C(G(x)),1)=−log(C(G(x)).

When using this cost function, correctly classified synthetic output data (shown as C(G(x))≈0) causes a rise in the cost function K_{G} that is to be minimized.

The model 33 that is trained by machine learning (e.g., the discriminator 40) with the parameters 50 determined in the training may be provided in act S16 and, for example, used in the method shown in FIG. 1.

As explained previously with reference to FIG. 4, it may be appropriate to identify the different change types using separate models 34, 35 that are trained by machine learning. A suitable training is explained below with additional reference to FIG. 8. Supervised training is intended to take place in the example. The training data 49 is used as original data in the context of the training. In addition, the training data sets 48 that are provided in act S14 each include processing data or comparison data 52. This is determined in each case from the training data 49 of the respective training data set 48 as a result of processing (e.g., noise reduction) and a respective desired result 53. The desired result 53 indicates whether or to what extent the respective processing data or comparison data 52 differs from the respective training data with respect to the change type that the model is to be trained to identify. The desired result 53 may be predetermined, for example, by manual assessment of the respective training data set by a medical specialist.

A convolutional neural network shown in FIG. 5 is used as a model in the example. The layers L.1 to L.10 are present twice (e.g., once in the subnetwork 58 shown and again in the subnetwork 59). The layer L.11 processes the output data from the respective layer L.10 of both the subnetwork 58 and the subnetwork 59 as input data. The subnetwork 58 or the layer L.1 thereof processes the training data 49 or, after the training, the reference data 12. The subnetwork 59 or the layer L.1 thereof processes in each case the processing data or comparison data 52 or, after the training, the comparison data 11.

The neural network in the example consists of convolutional layers, pooling layers, and fully connected layers. In the input layer L.1, there is a node for each pixel of the respective input data, each pixel having a channel (e.g., the respective intensity value). The input layer is followed by four convolutional layers L.2, L.4, L.6, L.8, each of the four convolutional layers being followed by a pooling layer L.3, L.5, L.7, L.9. For each of the convolutional layers, a 5×5 kernel (shown by “5×5 kernel”) with a padding of 2 (shown by “P:2”) and an increasing number of filters/convolution kernels (shown by “F:2”, “F:4” or “F:8”) is used. There are also four pooling layers L.3, L.5, L.7, L.9, of which the first three layers L.3, L.5, L.7 perform averaging over fields having a size of 4×4 and the final pooling layer L.9 performs a maximum selection over fields having a size of 2×2. FIG. 8 shows an additional layer L.10 that flattens the input images (e.g., the 8 images having a size of 4×4 are combined into a vector having 128 entries). However, this layer is not relevant for the actual calculation.

The last layers of the network are three fully connected layers L.11, L.12, L.13. The first fully connected layer has 256 input nodes and 60 output nodes, the second fully connected layer L. 12 has 60 input nodes and 10 output nodes, and the third fully connected layer L.13 has 10 input nodes and one output node, the output node forming the output layer of the complete machine learning model. The value of the node of the output layer corresponds to the probability that the processing data or comparison data 52 supplied to the subnetwork 59 differs from the training data 49 with respect to the trained change type concerned.

A database containing 500 medical images was used as training data for the training of the neural network. The processing data or comparison data 52 was generated by a processing algorithm as explained above. The desired result 53 was predetermined by radiologists as experts. In the example, the experts specified for each of the 500 data sets whether or to what extent the depiction of a vessel is correctly represented in the processing data or comparison data 52, normalized in the range 0 to 1 (basic truth). The database was divided into training data (e.g., 320 data sets), validation data (e.g., 80 datasets), and test data (e.g., 100 data sets).

For the purpose of the training, the back propagation algorithm was used based on a cost function that totals the quadratic deviation between the actual result 60 and the desired result 61 over the training data sets and then divides it by the number of training data sets.

Based on the validation set including 80 data sets and the associated remarks, the most powerful model was selected from a plurality of machine learning models (e.g., using different hyperparameters such as number of layers, size and number of kernels, padding, etc.). The specificity and sensitivity were determined based on the test set including 100 data sets and the associated remarks.

Although the invention is illustrated and described in detail above with reference to the example embodiments, the invention is not restricted to the examples disclosed herein, and other variations may be derived therefrom by a person skilled in the art without thereby departing from the scope of the invention.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for validating processing data, the method being computer-implemented and comprising:
receiving original data that is based on a medical image data acquisition;
receiving or determining the processing data, which is based on application of a main processing algorithm to the original data;
comparing comparison data that is the processing data or is based on the processing data, with reference data that is the original data or is based on application of a reference processing algorithm to the original data, using a comparison algorithm, such that a comparison result is determined;
evaluating a trigger condition, fulfillment of which depends on the comparison result; and
when the trigger condition is fulfilled:
outputting a notification to a user;
modifying a predetermined acquisition parameter for a subsequent image data acquisition;
providing new processing data, wherein the reference data is used as the new processing data, or the new processing data is determined by applying an alternative processing algorithm to the original data; or
any combination thereof,
wherein the comparison result describes in each case presence, extent, or the presence and the extent of a deviation of one or more deviation types, and
wherein the one or more deviation types include:
absence, interruption, truncated representation, or any combination thereof of at least one vessel;
absence of a depiction of a medical device;
absence of a depiction of an anatomical feature; or
any combination thereof in the comparison data relative to the reference data, in the reference data relative to the comparison data, or in the comparison data relative to the reference data and in the reference data relative to the comparison data.

2. The method of claim 1, wherein:
the main processing algorithm is configured to improve an image, change an image impression, or improve the image and change the image impression;
the original data comprises a plurality of two-dimensional image data sets, the main processing algorithm being or comprising a reconstruction of a three-dimensional or four-dimensional image data set from these two-dimensional image data sets; or
a combination thereof.

3. The method of claim 1, wherein the main processing algorithm and the reference processing algorithm, the alternative processing algorithm, or the main processing algorithm, the reference processing algorithm, and the alternative processing algorithm are implemented by respectively differing parameterization of a basic processing algorithm.

4. The method of claim 1, wherein the original data comprises a plurality of two-dimensional image data sets,
wherein the processing data takes the form of a three-dimensional or four-dimensional image data set that is reconstructed from the two-dimensional image data sets by the main processing algorithm, and
wherein the comparison data is or comprises two-dimensional image data sets that are determined by a respective forwards projection of the three-dimensional or four-dimensional image data set.

5. The method of claim 1, further comprising:
applying an evaluation algorithm to the comparison data, such that segments of the comparison data that depict a relevant anatomical feature, a medical device, or the relevant anatomical feature and the medical device are identified in each case; and
applying the evaluation algorithm to the reference data, such that segments of the reference data that depict the relevant anatomical feature, the medical device, or the relevant anatomical feature and the medical device are identified in each case,
wherein:
the comparison result depends on a comparison of a number, positions, dimensions, or any combination thereof of the segments in the comparison data with a number, positions, dimensions, or any combination thereof of the segments in the reference data;
a boundary outline for the respective segment is determined both in the reference data and in the comparison data, and the comparison result depends on a comparison of positions, dimensions, or the positions and the dimensions of the boundary outline in the comparison data with positions, dimensions, or the positions and the dimensions of the boundary outline in the reference data; or
a combination thereof.

6. The method of claim 1, wherein the comparison algorithm is or comprises a model that is trained by machine learning.

7. The method of claim 1, wherein the comparison algorithm comprises at least a first model and a second model that are trained by machine learning and, in each case, are configured to process the comparison data and the reference data as input data,
wherein the first model that is trained by machine learning determines a first intermediate result that relates to a presence, an extent, or the presence and the extent of a deviation of a first deviation type of the one or more deviation types between the comparison data and the reference data,
wherein the second model that is trained by machine learning determines a second intermediate result that relates to a presence, an extent, or the presence and the extent of a deviation of a second deviation type of the one or more deviation types between the comparison data and the reference data, the second deviation type being different than the first deviation type, and
wherein the comparison result depends on the first intermediate result and the second intermediate result.

8. The method of claim 6, wherein a discriminator is used as the model that is trained by machine learning, parameterization of the discriminator being based on a training of a generative adversarial network that comprises the discriminator.

9. The method of claim 7, wherein a discriminator is used as at least one model of the first model and the second model that are trained by machine learning, parameterization of the discriminator being based on a training of a generative adversarial network that comprises the discriminator.

10. The method of claim 1, further comprising receiving earlier original data that is based on a previous medical image data acquisition that took place prior to the medical image data acquisition,
wherein the predetermined acquisition parameter for the image data acquisition or an acquisition parameter for a subsequent image data acquisition is specified as a function of a further comparison result that is determined by the comparison algorithm by comparing current input data that corresponds to or is based on the original data with earlier input data that corresponds to or is based on the earlier original data.

11. The computer-implemented method of claim 1, wherein the comparison information, at least in the event that the trigger condition is fulfilled, relates to at least one segment of the comparison data in which the comparison data differs from the reference data, and
wherein the notification comprises segment information relating to the at least one segment.

12. A method for providing a model that is trained by machine learning for use as a comparison algorithm or sub-algorithm of the comparison algorithm, the method being computer-implemented and comprising:
receiving input training data that comprises a plurality of training data sets, the plurality of training data sets comprising training data that is based in each case on a medical image data acquisition, a simulation of the medical image data acquisition, or the medical image data acquisition and the simulation of the medical image data acquisition;
training a model based on the input training data, such that the model that is trained by machine learning is determined; and
providing the model that is trained by machine learning.

13. The method of claim 12, further comprising using the model that is trained by machine learning, the using of the model that is trained by machine learning comprising:
validating processing data, the validating comprising:
receiving original data that is based on a medical image data acquisition;
receiving or determining the processing data, which is based on application of a main processing algorithm to the original data;
comparing comparison data that is the processing data or is based on the processing data, with reference data that is the original data or is based on application of a reference processing algorithm to the original data, using a comparison algorithm, such that a comparison result is determined, the comparison algorithm being or comprising the model that is trained by machine learning;
evaluating a trigger condition, fulfillment of which depends on the comparison result; and
when the trigger condition is fulfilled:
outputting a notification to a user;
modifying a predetermined acquisition parameter for a subsequent image data acquisition;
providing new processing data, wherein the reference data is used as the new processing data, or the new processing data is determined by applying an alternative processing algorithm to the original data; or
any combination thereof,
wherein the comparison result describes in each case presence, extent, or the presence and the extent of a deviation of one or more deviation types, and
wherein the one or more deviation types include:
absence, interruption, truncated representation, or any combination thereof of at least one vessel;
absence of a depiction of a medical device;
absence of a depiction of an anatomical feature; or
any combination thereof in the comparison data relative to the reference data, in the reference data relative to the comparison data, or in the comparison data relative to the reference data and in the reference data relative to the comparison data.

14. An apparatus comprising:
a processor configured to validate processing data, the processor being configured to validate the processing data comprising the processor being configured to:
receive original data that is based on a medical image data acquisition;
receive or determine the processing data, which is based on application of a main processing algorithm to the original data;
compare comparison data that is the processing data or is based on the processing data, with reference data that is the original data or is based on application of a reference processing algorithm to the original data, using a comparison algorithm, such that a comparison result is determined;
evaluate a trigger condition, fulfillment of which depends on the comparison result; and
when the trigger condition is fulfilled:
output a notification to a user;
modify a predetermined acquisition parameter for a subsequent image data acquisition;
provide new processing data, wherein the reference data is used as the new processing data, or the new processing data is determined by application of an alternative processing algorithm to the original data; or
any combination thereof,
wherein the comparison result describes in each case presence, extent, or the presence and the extent of a deviation of one or more deviation types, and
wherein the one or more deviation types include:
absence, interruption, truncated representation, or any combination thereof of at least one vessel;
absence of a depiction of a medical device;
absence of a depiction of an anatomical feature; or
any combination thereof in the comparison data relative to the reference data, in the reference data relative to the comparison data, or in the comparison data relative to the reference data and in the reference data relative to the comparison data.

15. In a non-transitory computer-readable storage medium that stores instructions executable by one or more processors to validate processing data, the instructions comprising:
receiving original data that is based on a medical image data acquisition;
receiving or determining the processing data, which is based on application of a main processing algorithm to the original data;
comparing comparison data that is the processing data or is based on the processing data, with reference data that is the original data or is based on application of a reference processing algorithm to the original data, using a comparison algorithm, such that a comparison result is determined;
evaluating a trigger condition, fulfillment of which depends on the comparison result; and
when the trigger condition is fulfilled:
outputting a notification to a user;
modifying a predetermined acquisition parameter for a subsequent image data acquisition;
providing new processing data, wherein the reference data is used as the new processing data, or the new processing data is determined by applying an alternative processing algorithm to the original data; or
any combination thereof,
wherein the comparison result describes in each case presence, extent, or the presence and the extent of a deviation of one or more deviation types, and
wherein the one or more deviation types include:
absence, interruption, truncated representation, or any combination thereof of at least one vessel;
absence of a depiction of a medical device;
absence of a depiction of an anatomical feature; or
any combination thereof in the comparison data relative to the reference data, in the reference data relative to the comparison data, or in the comparison data relative to the reference data and in the reference data relative to the comparison data.
